(19) United States
(12) Patent Application Publication
(10) Pub. No.: US 2022/0214156 A1
(43) Pub. Date: Jul. 7, 2022

(54) OPTICAL COHERENCE TOMOGRAPHY APPARATUS, IMAGING METHOD, AND NON-TRANSITORY COMPUTER READABLE MEDIUM STORING IMAGING PROGRAM

(71) Applicant: NEC Corporation, Minato-ku, Tokyo (JP)

(72) Inventors: Atsufumi SHIBAYAMA, Tokyo (JP); Shigeru NAKAMURA, Tokyo (JP)

(73) Assignee: NEC Corporation, Minato-ku, Tokyo (JP)

(21) Appl. No.: 17/609,859

(22) PCT Filed: May 16, 2019
(86) PCT No.: PCT/JP2019/019559

§ 371 Date: Nov. 9, 2021

(51) Int. Cl. G01B 9/02091

(52) U.S. Cl. CPC G01B 9/02091; A61B 5/0066

(57) ABSTRACT

An optical coherence tomography apparatus (100) includes a wavelength sweeping laser light source (101), a branching and merging device (103) that branches a light beam emitted from the wavelength sweeping laser light source (101) into an object light beam and a reference light beam, an optical spectrum data generation unit (108) that generates information about wavelength dependence of an intensity difference between a plurality of interference light beams generated by interference between an object light beam scattered from a measurement object (200) and a reference light beam, and a control unit (109) that acquires structural data of the measurement object (200) in a depth direction on the basis of the information about wavelength dependence, and connects a plurality of pieces of structural data in the depth direction acquired at different positions in a scanning direction of the measurement object (200), wherein preliminary measurement is performed by causing the wavelength sweeping laser light source (101) to emit a light beam swept in a wavelength sweep range narrower than a wavelength sweep range in actual measurement, and the actual measurement is performed when it is determined that the measurement object (200) is at a position suitable for performing the actual measurement on the basis of results of the preliminary measurement.

### TECHNICAL FIELD

The present disclosure relates to an optical coherence tomography apparatus, an imaging method, and a non-transitory computer readable medium storing an imaging program.

### BACKGROUND ART

As a technique for performing tomographic imaging in the vicinity of a surface of a measurement object, there is an Optical Coherence Tomography (OCT) technology. In the OCT technology, tomographic imaging of the vicinity of the surface of a measurement object is performed by utilizing interference between a scattered light beam (hereinafter also referred to as a “backscattered light beam”) from the inside of the measurement object and a reference light beam when the measurement object is irradiated with a light beam. Recently, applications of the OCT technology to medical diagnosis and industrial product inspection have expanded.

In the OCT technology, a position of a part of the measurement object in an optical axis direction, which is a depth direction, where the object light beam is scattered in the measurement object, i.e., a light scattering point, is identified using interference between the object light beam applied and scattered from the measurement object and the reference light beam. By doing so, structural data spatially decomposed in the depth direction of the measurement object is obtained. In many cases, the object light beam is not reflected by 100% at the surface of the measurement object, and instead propagates to some extent to the inside of the measurement object and then is scattered backward. Therefore, the structural data spatially decomposed in the depth direction inside the measurement object part can be obtained. There are Time Domain (TD-OCT) and Fourier Domain (FD-OCT) methods in the OCT technology. The FD-OCT is more promising in terms of its high speed and high sensitivity. In the FD-OCT method, an interference light spectrum of a wide wavelength band is measured when the object light beam and the reference light beam interfere with each other, and the interference light spectrum is subjected to Fourier transformation to obtain the structural data in the depth direction. As a method for obtaining the interference light spectrum, there are the Spectral Domain (SD-OCT) method using a spectroscope and the Swept Source (SS-OCT) method using a light source for sweeping a wavelength.

Further, by scanning the object light beam in an in-plane direction perpendicular to the depth direction of the measurement object, tomographic structural data spatially decomposed in the in-plane direction and spatially decomposed in the depth direction can be obtained. In this manner, three-dimensional tomographic structural data of the measurement object can be obtained. Usually, the object light beam is scanned by a galvanomirror or the like, and the irradiation position of one object light beam is moved.

The OCT technology has been put into practical use as a tomography apparatus of the fundus oculi in an ophthalmic diagnosis, and its application as a non-invasive tomography apparatus to various parts of a living body is being studied.

FIG. 8 shows a typical configuration of an optical coherence tomography apparatus 500 of the SS-OCT method. A wavelength-swept optical pulse is generated from a wavelength sweeping laser light source 501. A light beam emitted from the laser light source 501 passes through a circulator 502 and is branched into an object light beam R1 and a reference light beam R2 in a branching and merging device 503. The object light beam R1 passes through a fiber collimator 504 and an irradiation optical system 505 composed of a scan mirror such as a galvanoscanner and a lens, and then is applied to a measurement object 200. Next, an object light beam R3 scattered in the measurement object 200 returns to the branching and merging device 503. On the other hand, the reference light beam R2 passes through a reference light mirror 506 and then returns to the branching and merging device 503. Thus, in the branching and merging device 503, the object light beam R3 scattered from the measurement object 200 and the reference light beam R4 reflected from the reference light mirror 506 interfere with each other to obtain interference light beams R5 and R6. Therefore, a ratio of intensity of the interference light beam R5 to that of the interference light beam R6 is determined by a phase difference between the object light beam R3 and the reference light beam R4. The interference light beam R5 passes through the circulator 502 and is then input to a two-input balanced photodetector 507, whereas the interference light beam R6 is directly input to the two-input balanced photodetector 507.

The ratio of the intensity of the interference light beam R5 to that of the interference light beam R6 changes according to a wavelength change of the light beam emitted from the wavelength sweeping laser light source 501. Thus, the wavelength dependence of the photoelectric conversion output of the balanced photodetector 507 can be measured as an interference light spectrum. By measuring the interference light spectrum and performing the Fourier transformation on the interference light spectrum, data indicating intensity of the backscattered light (the object light beam) at different positions in the depth direction (a Z direction) is obtained (hereinafter, an operation of obtaining the data indicating the intensity of the backscattered light beams (the object light beams) in the depth direction (the Z direction) at a certain position of the measurement object 200 will be referred to as a “A-scan”).

An irradiation position of an object light beam R1 is moved by the irradiation optical system 505, and the measurement object 200 is scanned. The irradiation optical system 505 repeatedly performs the A-scan operation while moving the irradiation position of the object light beam R1 in a scanning line direction (an X direction) and connecting the measurement results, thereby obtaining a map of the intensity of two-dimensional backscattered light beams (the object light beams) in the scanning line direction and the depth direction as the tomographic structural data (hereinafter, the operation of repeatedly performing the A-scan operation in the scanning line direction (the X direction) and connecting the measurement results is referred to as a “B-scan”).

Further, the irradiation optical system 505 repeatedly performs the B-scan operation while moving the irradiation position of the object light beam R1 not only in the scanning line direction but also in a direction (a Y direction) perpendicular to the scanning line, and connects the measurement results, thereby obtaining the three-dimensional tomographic structural data (hereinafter, the operation of repeatedly performing the B-scan operation in the direction (the Y direction) perpendicular to the scanning line and connecting the measurement results is referred to as a “C-scan”).

The interference light spectrum of N samples having a center wavelength λ₀ and a wavelength range Δλ is acquired in the A-scan, and the interference light spectrum is subject to discrete Fourier transformation, thereby obtaining the structural data in the depth direction using λ₀²/Δλ as a unit of length. When a period of the A-scan is ΔT and a speed of the object light beam R1 in the B-scan in the scanning line direction is V, the structural data (the tomographic structural data) in the scanning line direction having V/ΔT as a unit of length is obtained. That is, the positional accuracy of the three-dimensional tomographic structural data obtained by measurement by OCT is determined by operating conditions such as a wavelength sweeping laser light source and a galvanoscanner.

In the case where a measurement object is a living body, it is difficult to completely fix the measurement object for measurement, and it is therefore desirable to perform measurement at high speed. However, the measurement time corresponding to the time required for the A-scan involved in the wavelength sweeping of the wavelength sweeping laser light source 501 and the time required for the B-scan and the C-scan involved in the control of the irradiation optical system 505 is required. Further, since an increase in scanning speed results in a decrease in measurement accuracy, there is a limitation in increasing the speed.

Thus, in the case of measuring a non-stationary measurement object such as a living body, it is desirable to perform measurement at the timing when the measurement object is located in a measurable range. A method of using another sensor such as a camera may be used to detect the position of the measurement object; however, this causes an increase in apparatus size and cost due to the added sensor.

Patent Literature 1 describes detecting the position of the measurement object by performing the B-scan at a relatively high scan rate (a rate of acquiring a relatively rough image).

Patent Literature 2 describes acquiring a low resolution image of a part to check the overall position and shape over a wide range in the B-scan. To be specific, Patent Literature 2 describes acquiring an image of a part to check the overall position and shape by performing the B-scan using a light beam with a wavelength swept at a sweep interval greater (coarser) than a sweep interval in actual measurement in a sweep range greater than a sweep range in actual measurement.

### CITATION LIST

### Patent Literature

Patent Literature 1: Japanese Unexamined Patent Application Publication No. 2016-198280

Patent Literature 2: Japanese Unexamined Patent Application Publication No. 2019-025186

### SUMMARY OF INVENTION

### Technical Problem

However, in the optical coherence tomography apparatus disclosed in Patent Literature 1, the B-scan that detects the position of a measurement object is performed in the wavelength sweep range that is the same as the actual wavelength sweep range, the measurement time for detecting the position of a measurement object is not short enough.

Further, in the optical coherence tomography apparatus disclosed in Patent Literature 2, the B-scan for acquiring an image of a part to check the overall position and shape is performed in the wavelength sweep range that is larger than the actual wavelength sweep range. Thus, the measurement time for acquiring an image of a part to check the overall position and shape is not short enough.

Therefore, in Patent Literature 1 and Patent Literature 2, the position of a measurement object is not promptly detectable, which can cause a failure to detect the timing when the measurement object is at a position suitable for measurement. Thus, there are cases where measurement cannot be done at the timing when the measurement object is located in a measurable range.

An object of the present disclosure is to provide an optical coherence tomography apparatus, an imaging method, and a non-transitory computer readable medium storing an imaging program capable of detecting the position of a measurement object more promptly and performing measurement at appropriate timing.

### Solution to Problem

An optical coherence tomography apparatus according to a first aspect of the present disclosure includes a wavelength sweeping laser light source, a branching unit configured to branch a light beam emitted from the wavelength sweeping laser light source into an object light beam and a reference light beam, an irradiation unit configured to apply the object light beam output from the branching unit to different positions on a surface of a measurement object, a merging unit configured to cause interference of the object light beam scattered from the measurement object with the reference light beam and thereby generate a plurality of interference light beams, a measurement unit configured to generate information about wavelength dependence of an intensity difference between the plurality of interference light beams output from the merging unit, and a control unit configured to acquire structural data of the measurement object in a depth direction on the basis of the information about wavelength dependence of an intensity difference between the plurality of interference light beams generated by the measurement unit, control the irradiation unit to acquire a plurality of pieces of the structural data in the depth direction while moving a plurality of irradiation positions of the object light beam along a direction orthogonal to the depth direction of the measurement object, and connect the acquired plurality of pieces of the structural data in the depth direction, wherein preliminary measurement is performed by causing the wavelength sweeping laser light source to emit a light beam swept in a wavelength sweep range narrower than a wavelength sweep range in actual measurement, the control unit determines whether the measurement object is at a position suitable for performing the actual measurement on the basis of results of the preliminary measurement, and the actual measurement is performed when the control unit determines that the measurement object is at a position suitable for performing the actual measurement on the basis of results of the preliminary measurement.

An imaging method according to a second aspect of the present disclosure includes performing preliminary measurement by causing a wavelength sweeping laser light source to emit a light beam swept in a wavelength sweep range narrower than a wavelength sweep range in actual measurement, determining whether a measurement object is at a position suitable for performing the actual measurement on the basis of results of the preliminary measurement, and performing the actual measurement when it is determined that the measurement object is at a position suitable for performing the actual measurement on the basis of results of the preliminary measurement, each of the above operations being carried out by an optical coherence tomography apparatus.

A non-transitory computer readable medium storing an imaging program according to a third aspect of the present disclosure causes an optical coherence tomography apparatus to execute processing of performing preliminary measurement by emitting a light beam swept in a wavelength sweep range narrower than a wavelength sweep range in actual measurement, processing of determining whether a measurement object is at a position suitable for performing the actual measurement on the basis of results of the preliminary measurement, and processing of performing the actual measurement when it is determined that the measurement object is at a position suitable for performing the actual measurement on the basis of results of the preliminary measurement.

### Advantageous Effects of Invention

There are provided an optical coherence tomography apparatus, an imaging method, and a non-transitory computer readable medium storing an imaging program capable of detecting the position of a measurement object more promptly and performing measurement at appropriate timing.

### BRIEF DESCRIPTION OF DRAWINGS

FIG. 1 is a view showing an example of an optical coherence tomography apparatus according to the present disclosure;

FIG. 2 is a view shwoing an example of an optical coherence tomography apparatus according to a first example embodiment of the present disclosure;

FIG. 3 is a flowchart illustrating an example of an imagnig method according to the first example embodiment of the present disclosure;

FIG. 4 is a view showing an example of scanning with an object light beam in the optical coherence tomography apparatus according to the first example embodiment of the present disclosure;

FIG. 5 is a view showing an example of scanning with an object light beam in the optical coherence tomography apparatus according to the first example embodiment of the present disclosure;

FIG. 6 is a view showing an example of scanning with an object light beam in the optical coherence tomography apparatus according to the first example embodiment of the present disclosure;

FIG. 7 is a view showing an example of scanning with an object light beam in the optical coherence tomography apparatus according to the first example embodiment of the present disclosure; and

FIG. 8 is a view showing an example of an optical coherence tomography apparatus according to a related art.

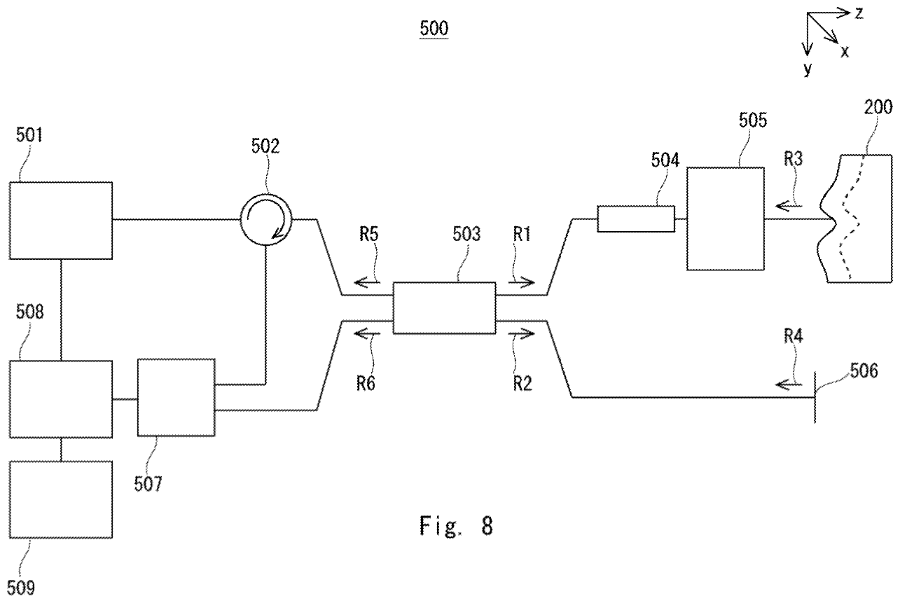

### DESCRIPTION OF EMBODIMENTS

Example embodiments of the present disclosure will be described below with reference to the drawings.

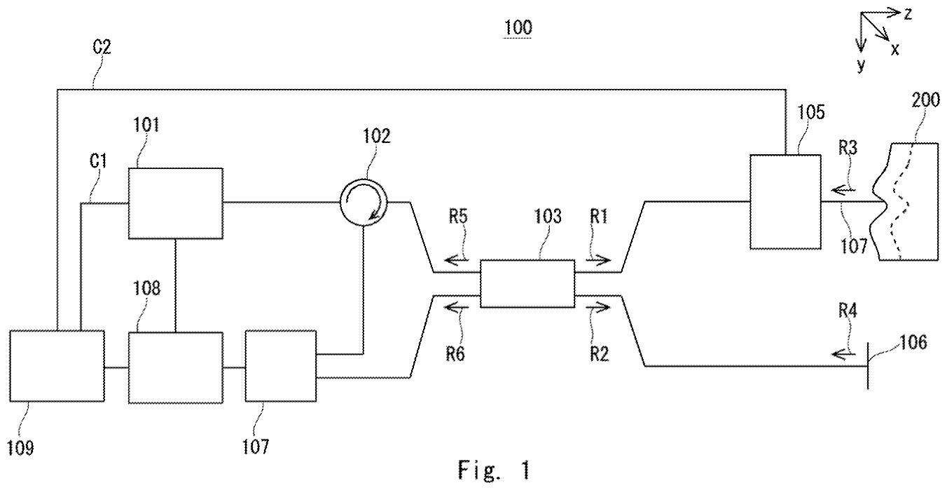

FIG. 1 is a view showing an example of an optical coherence tomography apparatus 100 according to the present disclosure. As shown in FIG. 1, the optical coherence tomography apparatus 100 includes a wavelength sweeping laser light source 101, a circulator 102, a branching and merging device 103 serving as a branching unit and a merging unit, an irradiation optical system 105 serving as an irradiation unit, a reference light mirror 106, a balanced photodetector 107 serving as a measurement unit, an optical spectrum data generation unit 108 serving as a measurement unit, a control unit 109, and so on.

A light beam emitted from the wavelength sweeping laser light source 101 passes through the circulator 102 and is branched into an object light beam R1 and a reference light beam R2 by the branching and merging device 103. The object light beam R1 output from the branching and merging device 103 passes through the irradiation optical system 105, and then is applied to a measurement object 200. To be specific, the irradiation optical system 105 applies the object light beam R1 to different positions of the measurement object 200 in the X-Y plane and thereby scans a certain range of the measurement object 200.

The object light beam R1 applied to the measurement object 200 is scattered backward (in a direction opposite to an irradiation direction of the object light beam R1) from the measurement object 200. Then, an object light beam (a backscattered light beam) R3 scattered from the measurement object 200 passes through the irradiation optical system 105, and then returns to the branching and merging device 103.

The reference light beam R2 output from the branching and merging device 103 is reflected by the reference light mirror 106 and then returns to the branching and merging device 103.

In the branching and merging device 103, the object light beam R3 and the reference light beam R4 interfere with each other to obtain an interference light beam R5 and an interference light beam R6.

The interference light beam R5 passes through the circulator 102 and is then input to the balanced photodetector 107, whereas the interference light beam R6 is directly input to the balanced photodetector 107. Then, information about a difference in intensity between the interference light beam R5 and the interference light beam R6 is input from the balanced photodetector 107 to the optical spectrum data generation unit 108.

Then, the balanced photodetector 107 photoelectrically converts an intensity difference I(k) between the interference light beam R5 and the interference light beam R6. The balanced photodetector 107 inputs information about the intensity difference I(k) between the interference light beam R5 and the interference light beam R6 obtained by the photoelectric conversion to the optical spectrum data generation unit 108.

The optical spectrum data generation unit 108 generates interference light spectrum data on the basis of a signal input from the wavelength sweeping laser light source 101 and a signal input from the balanced photodetector 107. Specifically, information about a wavelength change of the light beam emitted from the wavelength sweeping laser light source 101 is input from the wavelength sweeping laser light source 101 to the optical spectrum data generation unit 108. Further, the information about the intensity difference I(k) between the interference light beam R5 and the interference light beam R6 is input from the balanced photodetector 107 to the optical spectrum data generation unit 108. The optical spectrum data generation unit 108 generates the interference light spectrum data (the wavelength dependence of the intensity difference I(k) between the interference light beam R5 and the interference light beam R6) on the basis of the information about the wavelength change of the light beam emitted from the wavelength sweeping laser light source 101 and the information about the intensity difference I(k) between the interference light beam R5 and the interference light beam R6.

The control unit 109 acquires structural data in the depth direction (the Z direction) of the measurement object 200 on the basis of the interference light spectrum data generated by the optical spectrum data generation unit 108. Further, the control unit 109 controls the irradiation optical system 105 to acquire a plurality of structural data pieces in the depth direction while moving a plurality of irradiation positions of the object light beam R1 along a direction (at least one of an X direction and a Y direction) orthogonal to the depth direction (the Z direction) of the measurement object 200. In other words, the control unit 109 acquires a plurality of structural data pieces in the depth direction at different positions of the measurement object 200 along at least one of the X direction and the Y direction. Then, the control unit 109 connects the acquired plurality of structural data pieces in the depth direction to acquire two-dimensional or three-dimensional tomographic structural data.

Further, the control unit 109 controls each unit of the optical coherence tomography apparatus 100 so as to perform a measurement process including first measurement as preliminary measurement and secnod measurement as actual measurement. The first measurement is performed to detect the position of the measurement object 200. The second measurement is performed to obtain three-dimensional tomographic structural data of the measurement object 200 with desired accuracy.

To be specidic, in the first measurement (preliminary measurement), the control unit 109 controls the wavelength sweeping laser light source 101 so as to emit a light beam that is swept in a wavelength sweep range that is narrower than a wavelength sweep range in the second measurement (actual measurement). The time for the A-scan in the first measurement is thereby reduced, so that the first measurement is performed at a higher speed than the second measurement. The position of the measurement object 200 is thereby detected more promptly.

Narrowing the wavelength sweep range of the wavelength sweeping laser light source 101 leads to degradation of the measurement accuracy in the depth direction (the Z direction). However, since the first measurement aims at detecting the position of the measurement object 200, the wavelength sweep range can be narrowed down to the measurement accuracy required for detection of the position.

On the basis of results of the first measurement, the control unit 109 determines whether the measurement object 200 is at a position suitable for performing the second measurement. When the control unit 109 determines that the measurement object 200 is at a position suitable for performing the second measurement on the basis of results of the first measurement, the control unit 109 controls each unit of the optical coherence tomography apparatus 100 so as to perform the second measurement.

The optical coherence tomography apparatus 100 according to the present disclosure described above narrows the wavelength sweep range of the wavelength sweeping laser light source 101 down to the measurement accuracy required for detecting the position of the measurement object 200 in the first measurement (preliminary measurement), and thereby reduces the time for the A-scan in the first measurement. This enables more prompt detection of the position of the measurement object 200 and avoids a failure to detect the timing when the measurement object 200 is at a position suitable for measurement. The optical coherence tomography apparatus 100 capable of detecting the position of the measurement object 200 more promptly and performing measurement at appropriate timing is thereby provided.

### First Example Embodiment

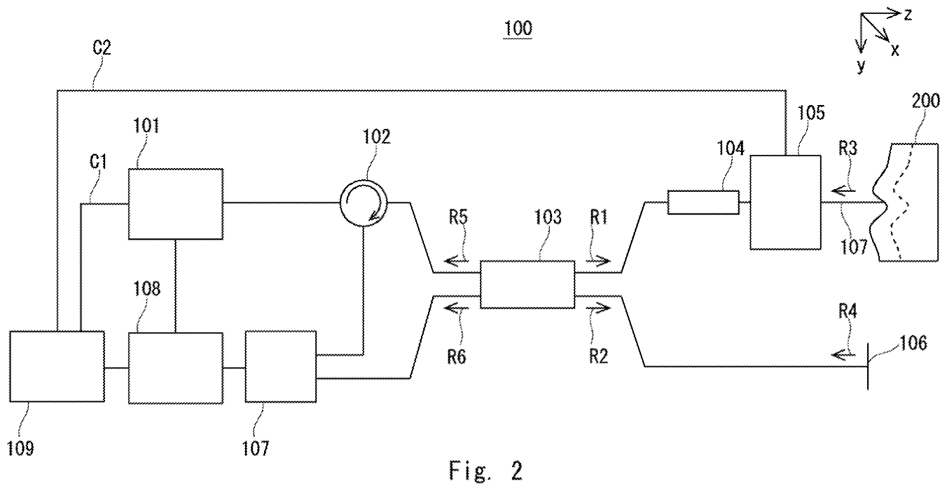

An optical coherence tomography apparatus 100 according to a first example embodiment of the present disclosure is described hereinafter. FIG. 2 is a view showing an example of the optical coherence tomography apparatus 100 according to the first example embodiment. As shown in FIG. 2, the optical coherence tomography apparatus 100 includes a wavelength sweeping laser light source 101, a circulator 102, a branching and merging device 103 serving as a branching unit and a merging unit, a fiber collimator 104, an irradiation optical system 105 serving as an irradiation unit, a reference light mirror 106, a balanced photodetector 107 serving as a measurement unit, an optical spectrum data generation unit 108 serving as a measurement unit, a control unit 109, and so on.

The wavelength sweeping laser light source 101 generates a wavelength-swept optical pulse according to a wavelength sweeping control signal C1 that is input from the control unit 109. To be specific, the wavelength sweeping laser light source 101 operates in a first operation mode or a second operation mode according to the wavelength sweeping control signal C1. The first operation mode is an operation mode in which the wavelength sweep range is narrower than that in the second operation mode. In contrast, the second operation mode is an operation mode in which the wavelength sweep range is wider than that in the first operation mode.

A light beam emitted from the wavelength sweeping laser light source 101 passes through the circulator 102 and is branched into an object light beam R1 and a reference light beam R2 by the branching and merging device 103. A first object light beam R1 output from the branching and merging device 103 passes through the fiber collimator 104 and the irradiation optical system 105, and then is applied to the measurement object 200.

To be specific, the irradiation optical system 105 applies the object light beam R1 to different positions of the measurement object 200 in the X-Y plane according to a scan control signal C2 that is input from the control unit 109, and thereby scans a certain range of the measurement object 200. To be more specific, the irradiation optical system 105 operates in the first operation mode or the second operation mode according to the scan control signal C2. The first operation mode is an operation mode in which the scanning time is shorter than that in the second operation mode. In contrast, the second operation mode is an operation mode in which the scanning time is longer than that in the first operation mode.

The object light beam R1 applied to the measurement object 200 is scattered backward (in a direction opposite to an irradiation direction of the object light beam R1) from the measurement object 200. Then, an object light beam (a backscattered light beam) R3 scattered from the measurement object 200 passes through the irradiation optical system 105 and the fiber collimator 104, and then returns to the branching and merging device 103.

The reference light beam R2 output from the branching and merging device 103 is reflected by the reference light mirror 106 and then returns to the branching and merging device 103.

In the branching and merging device 103, the object light beam R3 scattered from the measurement object 200 and the reference light beam R4 reflected from the reference light mirror 106 interfere with each other to obtain an interference light beam R5 and an interference light beam R6.

The interference light beam R5 passes through the circulator 102 and is then input to the balanced photodetector 107, whereas the interference light beam R6 is directly input to the balanced photodetector 107. Then, information about a difference in intensity between the interference light beam R5 and the interference light beam R6 is input from the balanced photodetector 107 to the optical spectrum data generation unit 108. Note that the balanced photodetector 107 is a photodetector in which two photodiodes are connected in series, and a connection between the photodiodes is an output (a differential output). A band of the balanced photodetector 107 is 1 GHz or less.

The interference between the object light beam R3 with a wavelength λ and a wave number k (=2π/λ) and the reference light beam R4 is described hereinafter. An optical path length of the reference light beam R2 that is branched by the branching and merging device 103, reflected by the reference light mirror 106 and returns to the branching and merging device 103 is L_{R}. On the other hand, an optical path length of the object light beam R1 that is branched by the branching and merging device 103, scattered backward at one light scattering point of the measurement object 200 and returns to the branching and merging device 103 is L_{S}=L_{R}+z₀. z₀ indicates the position in the depth direction (the Z direction) of the object light beam R1 scattered on the measurement object 200. In the branching and merging device 103, the object light beam R3 and the reference light beam R4 are superimposed with a phase difference kz₀+φ and interfere with each other. φ is a constant number not depending on k or z₀. When the amplitudes of the object light beam R3 and the reference light beam R4 that interfere with each other in the branching and merging device 103 are E_{S} and E_{R}, respectively, the intensity difference between the interference light beam R5 and the interference light beam R6 is represented as:

I(k)∝E_{S}·E_{R}·cos(kz₀+ϕ) [Expression 1]

Then, the balanced photodetector 107 photoelectrically converts the intensity difference I(k) between the interference light beam R5 and the interference light beam R6. The balanced photodetector 107 inputs information about the intensity difference I(k) between the interference light beam R5 and the interference light beam R6 obtained by the photoelectric conversion to the optical spectrum data generation unit 108.

The optical spectrum data generation unit 108 generates interference light spectrum data on the basis of information about a wavelength change of the light beam emitted from the wavelength sweeping laser light source 101 and information about the intensity difference I(k) between the interference light beam R5 and the interference light beam R6. The information about a wavelength change of the light beam emitted from the wavelength sweeping laser light source 101 is input from the wavelength sweeping laser light source 101 to the optical spectrum data generation unit 108. The information about the intensity difference I(k) between the interference light beam R5 and the interference light beam R6 is input from the balanced photodetector 107 to the optical spectrum data generation unit 108. A modulation with a period of 2π/z₀ appears in the interference light spectrum data I(k) obtained by performing measurement with wave numbers k₀−Δk/2 to k₀+Δk/2.

To be more specific, in the case where the measurement object 200 is a mirror, there is one light scattering point position of the object light beam R1. However, in the case where the measurement object 200 is not a mirror, the object light beam R1 applied to the measurement object 200 propagates to some extent to the inside of the measurement object 200 and then is sequentially scattered backward, and therefore light scattering points of the object light beam R1 are generally distributed in the range from the surface to a certain depth. In the case where the light scattering points of the object light beam R1 are distributed in the depth direction from z₀−Δz to z₀+Δz, modulations with periods 2π/(z₀−Δz) to 2π/(z₀+Δz) appear, superimposed with one another, in the interference light spectrum I(k).

The optical spectrum data generation unit 108 inputs the generated interference light spectrum I(k) to the control unit 109.

The control unit 109 performs Fourier transformation on the interference light spectrum data I(k). An amplitude J(z) obtained by Fourier transformation on the interference light spectrum data I(k) is

J(z)=|∫I(k)e^{izk}dk|∝δ(z−z₀)+δ(z+z₀) [Expression 2]

J(z) indicates a peak (hereinafter referred to as a “δ function peak”) obserted in a δ function (delta function) at z=z₀ (and z=−z₀) that reflects the light scattering point position z₀ of the object light beam R3.

In this procedure, data indicating the intensity (amplitude J(z)) of the object light beam R3 scattered backward at different positions of the measurement object 200 in the depth direction (the Z direction) is obtained by interfering the object light beam R3 with the common reference light beam R4 (hereinafter, the operation of obtaining data indicating the intensity of a backscattered light beam (object light beam) in the depth direction (the Z direction) at a certain position of the measurement object 200 will be referred to as a “A-scan”).

Further, the control unit 109 controls each unit of the optical coherence tomography apparatus 100.

For example, the control unit 109 controls the irradiation optical system 105 so as to apply the object light beam R1 to different positions of the measurement object 200 in the X-Y plane. Further, the control unit 109 controls the period and the speed for scanning the measurement object 200 by the irradiation optical system 105.

Further, the control unit 109 controls the irradiation optical system 105 to repeatedly perform the A-scan operation while moving the irradiation position of the object light beam R1 in a scanning line direction (at least one of the X direction and the Y direction). Then, the control unit 109 connects a plurality of measurement results obtained by repeatedly performing the A-scan operaiton while moving the irradiation position of the object light beam R1 in the scanning line direction. The control unit 109 thereby generates two-dimensional tomographic structural data (hereinafter, the operation of repeatedly performing the A-scan operation in the scanning line direction (at least one of the X direction and the Y direction) and connecting the measurement results is referred to as a “B-scan”)

Further, the control unit 109 controls the irradiation optical system 105 to repeatedly perform the B-scan operation while moving the irradiation position of the object light beam R1 not only in the scanning line direction but also in a direction perpendicular to the scanning line. Then, the control unit 109 connects a plurality of measurement results obtained by repeatedly performing the B-scan operaiton while moving the irradiation position of the object light beam R1 in the scanning line direction and in the direction perpendicular to the scanning line. The control unit 109 thereby generates three-dimensional tomographic structural data in X, Y, Z directions (hereinafter, the operation of repeatedly performing the B-scan operation in the scanning line direction and in the direction perpendicular to the scanning line and connecting the measurement results is referred to as a “C-scan”).

Furthermore, the control unit 109 performs processing of connecting a purality of three-dimensional tomographic structural data obtained by scanning with the object light beam R1.

The control unit 109 further controls each unit of the optical coherence tomography apparatus 100 so as to perform a measurement process including the first measurement and the secnod measurement. The first measurement is performed to detect the position of the measurement object 200. The second measurement is performed to obtain three-dimensional tomographic structural data of the measurement object 200 with desired accuracy.

To be specific, in the first measurement, the control unit 109 controls the wavelength sweeping laser light source 101 and the irradiation optical system 105 so as to operate in the first operation mode.

In the second measurement, the control unit 109 controls the wavelength sweeping laser light source 101 and the irradiation optical system 105 so as to operate in the second operation mode.

To be more specific, the first operation mode is an operation mode in which the wavelength sweep range of the wavelength sweeping laser light source 101 is narrower than that in the second operation mode. The time for the A-scan in the first measurement is thereby reduced, so that the first measurement is performed at a higher speed than the second measurement. The position of the measurement object 200 is thereby detected more promptly.

Narrowing the wavelength sweep range of the wavelength sweeping laser light source 101 leads to degradation of the measurement accuracy in the depth direction (the Z direction). However, since the first measurement aims at detecting the position of the measurement object 200, the wavelength sweep range can be narrowed down to the measurement accuracy required for detection of the position.

Further, the first operation mode is an operation mode in which the time for the irradiation optical system 105 to perform scanning with the object light beam R1 is shorter than that in the second operation mode. The time for the B-scan and the C-scan is thereby reduced, so that the first measurement is performed at a higher speed than the second measurement.

To be specific, in order to make the scanning time of the irradiation optical system 105 in the first operation mode shorter than that in the second operation mode, the control unit 109 controls the irradiation optical system 105 so as to achieve at least one of the following (1) to (3).

(1) The scanning speed of the irradiation optical system 105 is set higher than that in the second operation mode.
(2) The scanning range of the irradiation optical system 105 is set narrower than that in the second operation mode.
(3) The scanning in the irradiation optical system 105 is performed along a Lissajous curve (hereinafter referred to as a Lissajous scan).

With regard to the above (1), increasing the scanning speed of the irradiation optical system 105 leads to degradation of the measurement accuracy. However, the first measurement aims at detecting the position of the measurement object 200. Thus, by increasing the scanning speed up to the measurement accuracy required for detection of the position in the first measurement, the time for the B-scan and the C-scan is reduced. This allows more prompt detection of the position of the measurement object 200.

With regard to the above (2), narrowing the scanning range of the irradiation optical system 105 leads to a case where the whole body of the measurement object 200 cannot be measured. However, since the first measurement aims at detecting the position of the measurement object 200, it is not always necessary to measure the whole body. Thus, by narrowing the scanning range of the irradiation optical system 105 down to the range required for detecting the position of the measurement object 200 in the first measurement, the time for the B-scan and the C-scan is reduced. This allows more prompt detection of the position of the measurement object 200.

With regard to the above (3), a scan mirror such as a galvanos canner is generally used for beam scanning in the irradiation optical system 105. However, there is a physical limitation to a change in speed since it is necessary to physically control the angle of the mirror. The Lissajous scan has a feature that a change in scanning speed is smaller compared with a Raster scan, which allows an increase in scanning speed. In other words, by performing scanning along a trajectory with a smaller change in scanning speed in the first measurement, the time for the B-scan and the C-scan is reduced. This allows more prompt detection of the position of the measurement object 200.

Then, the control unit 109 determines whether the measurement object 200 is at a position suitable for performing the second measurement on the basis of results of the first measurement. When the control unit 109 determines that the measurement object 200 is at a position suitable for performing the second measurement on the basis of results of the first measurement, the control unit 109 controls each unit of the optical coherence tomography apparatus 100 so as to perform the second measurement.

The control unit 109 includes a CPU (Central Processing Unit), not shown, and a storage unit, not shown. Then, the CPU executes a program stored in the storage unit, thereby implementing all the processing of the control unit 109.

The programs stored in the respective storage units of the control unit 109 include codes for implementing processing of the control unit 109 by being executed by the CPU. The storage unit includes, for example, any storage device capable of storing this program and various kinds of information used for processing in the control unit 109. The storage device is, for example, a memory or the like.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (Read Only Memory), CD-R, CD-R/W, and semiconductor memories (such as mask ROM, PROM (Programmable ROM), EPROM (Erasable PROM), flash ROM, RAM (Random Access Memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

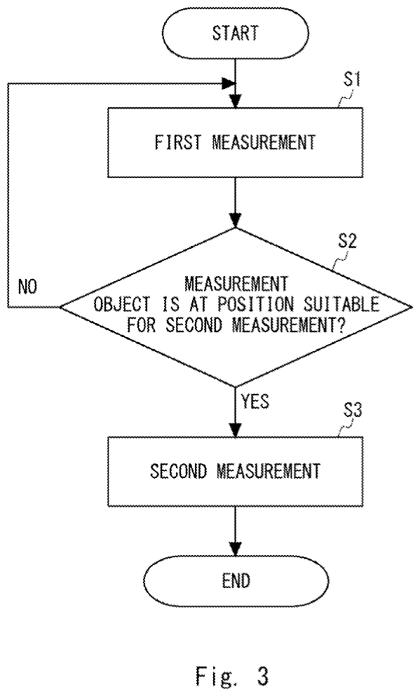

An imaging method according to the first example embodiment is described hereinafter with reference to FIG. 3. FIG. 3 is a flowchart illustrating an example of the imaging method according to the first example embodiment. As shown in FIG. 3, the imaging method according to the first example embodiment includes Step S1 in which the optical coherence tomography apparatus 100 performs the first measurement in order to detect the position of the measurement object 200, Step S2 in which the control unit 109 determines whether the measurement object 200 is at a position suitable for performing the second measurement, and Step S3 in which the optical coherence tomography apparatus 100 performs the second measurement in order to obtain three-dimensional tomographic structural data of the measurement object 200 with desired accuracy.

First, as shown in FIG. 3, the control unit 109 controls each part of the optical coherence tomography apparatus 100 so as to perform the first measurement (Step S1). The first measurement is a measurement for detecting the position of the measurement object 200, and the speed of measurement is higher than that in the second measurement.

To be specific, the control unit 109 inputs the wavelength sweeping control signal C1 to the wavelength sweeping laser light source 101 and controls the wavelength sweeping laser light source 101 so as to operate in the first operation mode. The first operation mode is an operation mode in which the wavelength sweep range is narrower than that in the second operation mode. Thus, wavelength sweeping is done earlier in the first operation mode than in the second operation mode. As a result, the time for the A-scan is reduced, which allows the measurement to be done at higher speed.

Further, the control unit 109 inputs the scan control signal C2 to the irradiation optical system 105 and controls the irradiation optical system 105 so as to operate in the first operation mode. The first operation mode is an operation mode in which the time to perform scanning with the object light beam R1 is shorter than that in the second operation mode. Thus, scanning is done earlier in the first operation mode than in the second operation mode. As a result, the time for the B-scan and the C-scan is reduced, which allows the measurement to be done at higher speed.

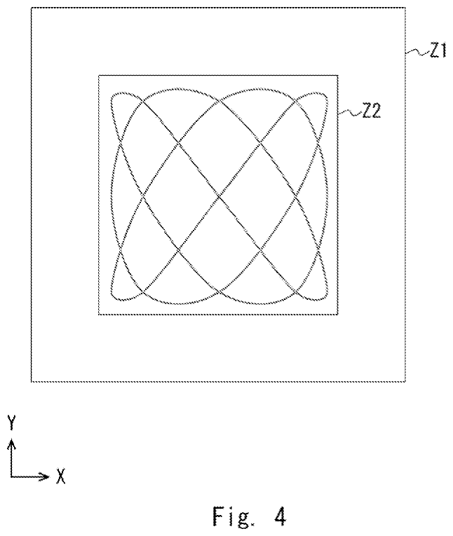

FIG. 4 shows an example of beam scanning of the irradiation optical system 105 in the first operation mode. FIG. 4 shows the trajectory of the Lissajous scan that performs scanning along the Lissajous curve which is represented by:

X=COS(3θ)

Y=SIN(4θ)

in a measurement range Z2 in the first operation mode, which is narrower than a measurement range Z1 in the X-Y plane in the second operation mode.

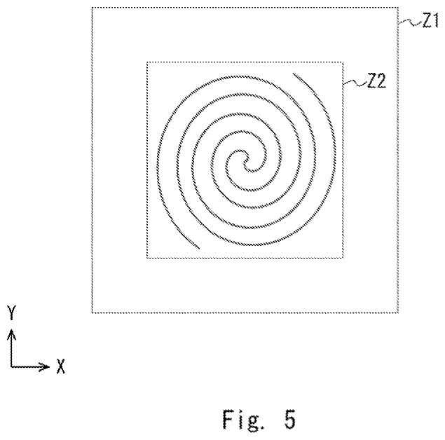

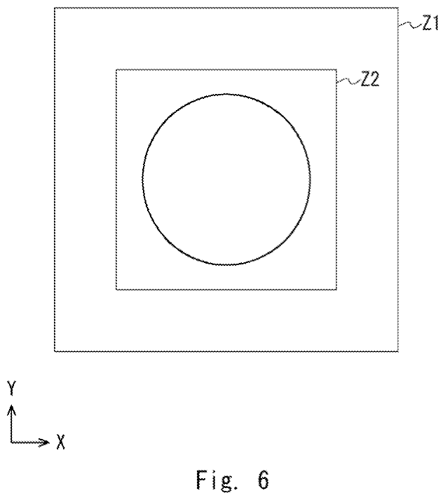

Note that beam scanning in the first operation mode is not limited to the Lissajous scan, and another method may be used as long as the scanning time is short and the position of the measurement object 200 is detectable. For example, a scan may be performed along a spiral trajectory as shown in FIG. 5 or a circular trajectory as shown in FIG. 6.

Next, the control unit 109 refers to the three-dimensional tomographic structural data of the measurement object 200 generated in the first measurement and determines whether the measurement object 200 is at a position suitable for performing the second measurement (Step S2). For example, when the measurement object 200 occupies a certain percentage of the X-Y plane at a certain depth (for example, z=z₁) of the three-dimensional tomographic structural data of the measurement object 200, the control unit 109 determines that the measurement object 200 is at a position suitable for performing the second measurement. For example, assuming that the whole of the measurement range Z2 in the first operation mode on the X-Y plane is 100%, the control unit 109 determines that the measurement object 200 is at a position suitable for performing the second measurement when the percentage of the measurement range Z2 occupied by the measurement object 200 is 70% or more.

In Step S2, when the measurement object 200 is not located at a position suitable for performing the second measurement (No in Step S2), the control unit 109 returns to the processing of Step S1.

In Step S2, when the measurement object 200 is located at a position suitable for performing the second measurement (Yes in Step S2), the control unit 109 controls each unit of the optical coherence tomography apparatus 100 so as to perform the second measurement (Step S3). The second measurement is a measurement for obtaining the three-dimensional tomographic structural data of the measurement object 200 with desired accuracy, and the accuracy of measurement is higher than that in the first measurement.

To be specific, the control unit 109 inputs the wavelength sweeping control signal C1 to the wavelength sweeping laser light source 101 and controls the wavelength sweeping laser light source 101 so as to operate in the second operation mode. The second operation mode is an operation mode in which the wavelength sweep range is wider than that in the first operation mode. Thus, the measurement accuracy in the depth direction (the Z direction) in the second operation mode is higher than that in the first operation mode.

Further, the control unit 109 inputs the scan control signal C2 to the irradiation optical system 105 and controls the irradiation optical system 105 so as to operate in the second operation mode. In the second operation mode, a measurement is performed with measurement accuracy and in a measurement range necessary for obtaining desired three-dimensional tomographic structural data. To be specific, a scan is performed with the object light beam R1 at a scanning speed that achieves the necessary measurement accuracy and in a scanning range that achieves the necessary measurement range. In other words, the second operation mode is an operation mode in which the time of scanning with the object light beam R1 is longer than that in the first operation mode.

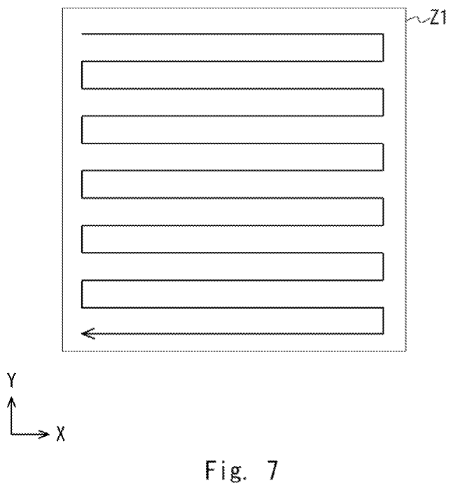

FIG. 7 shows an example of beam scanning of the irradiation optical system 105 in the second operation mode. In FIG. 7, beam scanning is perfomred by the Raster scan on the whole of the measurement range Z1 in the X-Y plane in the second operation mode. The Raster scan allows the measurement range to be uniformly irradiated with the object light beam R1, so that the accuracy of measurement is higher than that of the Lissajous scan or the like in the first operation mode.

As described above, the optical coherence tomography apparatus 100 according to the first example embodiment repeatedly performs the first measurement at high speed until it is determined that the measurement object 200 is at a position suitable for performing the second measurement. After it is determined that the the measurement object 200 is at a position suitable for performing the second measurement, the optical coherence tomography apparatus 100 performs the second measurement and obtains desired three-dimensional tomographic structural data.

Specific numerical examples in the first measurement and the second measurement are as follows.

In the first operation mode, the wavelength sweeping laser light source 101 generates an optical pulse whose wavelength increases from 1275 nm to 1325 nm with a duration of 5 μs, and it repeatedly generates this optical pulse at 200 kHz per 5 μs.

In the second operation mode, the wavelength sweeping laser light source 101 generates an optical pulse whose wavelength increases from 1250 nm to 1350 nm with a duration of 10 μs, and it repeatedly generates this optical pulse at 100 kHz per 10 μs.

In the first operation mode, the irradiation optical system 105 performs the Lissajous scan shown in FIG. 4 at a speed of 50 ms per period on the measurement range Z2 with a size of 10 mm×10 mm.

In the second operation mode, the irradiation optical system 105 performs the Raster scan shown in FIG. 6 at a speed of 5 m/s on the measurement range Z2 with a size of 15 mm×15 mm.

Thus, in the first measurement, a single measurement (a single Lissajous scan) is done in 50 ms, and the measurement (Lissajous scan) is performed repeatedly at a rate of 20 times per second. In a single measurement (a single Lissajous scan) in the first measurement, the A-scan is performed 10000 (50 ms/5 μs=10000) times in the measurement range of 10 mm×10 mm, so that 10000 points of data in the depth direction are obtained.

On the other hand, in the second measurement, the single B-scan is completed in 3 ms (15 mm/(5 m/s)=3 ms), and the A-scan is performed 300 (3 ms/10 μs=300) times in the scanning range of 15 mm. To perform the B-scan and the C-scan with the same measurement accuracy, the A-scan is performed 90000 (300×300=90000) times in the measurement range of 15 mm×15 mm in a single measurement in the second measurement. As a result, the second measurement is done in 0.9 s (10 μs (the optical pulse generation period of the wavelength sweeping laser light source 101=the time for performing the A-scan once)×90000 times=0.9 s), so that 90000 points of data in the depth direction are obtained.

Thus, the first measurement is 18 times faster than the second measurement, and the measurement accuracy of the second measurement is 9 times higher than that of the first measurement.

The optical coherence tomography apparatus 100 according to the first example embodiment described above narrows the wavelength sweep range of the wavelength sweeping laser light source 101 down to the measurement accuracy required for detecting the position of the measurement object 200 in the first measurement (preliminary measurement) and thereby reduces the time for the A-scan in the first measurement. This enables more prompt detection of the position of the measurement object 200 and avoids a failure to detect the timing when the measurement object 200 is at a position suitable for measurement. The optical coherence tomography apparatus 100, an imaging method, and a non-transitory computer readable medium storing an imaging program capable of detecting the position of a measurement object 200 more promptly and performing measurement at appropriate timing are thereby provided.

Further, in the first measurement (preliminary measurement), the irradiation optical system 105 scans a range narrower than a scanning range in the second measurement (actual measurement). Narrowing the scanning range of the irradiation optical system 105 leads to a case where the whole body of the measurement object 200 cannot be measured. However, since the first measurement aims at detecting the position of the measurement object 200, it is not always necessary to measure the whole body. Thus, by narrowing the scanning range of the irradiation optical system 105 down to the range required for detecting the position of the measurement object 200 in the first measurement, the time for the B-scan and the C-scan is reduced. This allows more prompt detection of the position of the measurement object 200.

Further, in the first measurement, the irradiation optical system 105 performs scanning at a higher speed than the speed of scanning in the second measurement. Increasing the scanning speed of the irradiation optical system 105 leads to degradation of the measurement accuracy. However, the first measurement aims at detecting the position of the measurement object 200. Thus, by increasing the scanning speed up to the measurement accuracy required for detection of the position in the first measurement, the time for the B-scan and the C-scan is reduced. This allows more prompt detection of the position of the measurement object 200.

Further, in the first measurement, the irradiation optical system 105 performs scanning along a trajectory with a smaller change in speed than a change in speed of scanning in the second measurement. A scan mirror such as a galvanoscanner is generally used for beam scanning in the irradiation optical system 105. However, there is a physical limitation to a change in speed since it is necessary to physically control the angle of the mirror. By performing scanning along a trajectory with a smaller change in scanning speed in the first measurement, the time related to the B-scan and the C-scan is reduced. This allows more prompt detection of the position of the measurement object 200.

The whole or part of the example embodiments disclosed above can be described as, but not limited to, the following supplementary notes.

### Supplementary Note 1

An optical coherence tomography apparatus comprising:

a wavelength sweeping laser light source;

a branching unit configured to branch a light beam emitted from the wavelength sweeping laser light source into an object light beam and a reference light beam;

an irradiation unit configured to apply the object light beam output from the branching unit to different positions on a surface of a measurement object;

a merging unit configured to cause interference of the object light beam scattered from the measurement object with the reference light beam and generate a plurality of interference light beams;

a measurement unit configured to generate information about wavelength dependence of an intensity difference between the plurality of interference light beams output from the merging unit; and

a control unit configured to acquire structural data of the measurement object in a depth direction on the basis of the information about wavelength dependence of an intensity difference between the plurality of interference light beams generated by the measurement unit, control the irradiation unit to acquire a plurality of pieces of the structural data in the depth direction while moving a plurality of irradiation positions of the object light beam along a direction orthogonal to the depth direction of the measurement object, and connect the acquired plurality of pieces of the structural data in the depth direction, wherein

preliminary measurement is performed by causing the wavelength sweeping laser light source to emit a light beam swept in a wavelength sweep range narrower than a wavelength sweep range in actual measurement,

the control unit determines whether the measurement object is at a position suitable for performing the actual measurement on the basis of results of the preliminary measurement, and

the actual measurement is performed when the control unit determines that the measurement object is at a position suitable for performing the actual measurement on the basis of results of the preliminary measurement.

### Supplementary Note 2

The optical coherence tomography apparatus according to Supplementary note 1, wherein in the preliminary measurement, the irradiation unit scans a range narrower than a range of scanning in the actual measurement.

### Supplementary Note 3

The optical coherence tomography apparatus according to Supplementary note 1 or 2, wherein in the preliminary measurement, the irradiation unit performs scanning at a higher speed than a speed of scanning in the actual measurement.

### Supplementary Note 4

The optical coherence tomography apparatus according to any one of Supplementary notes 1 to 3, wherein in the preliminary measurement, the irradiation unit performs scanning along a trajectory with a smaller change in speed than a change in speed of scanning in the actual measurement.

### Supplementary Note 5

An imaging method comprising:

performing preliminary measurement by causing a wavelength sweeping laser light source to emit a light beam swept in a wavelength sweep range narrower than a wavelength sweep range in actual measurement;

determining whether a measurement object is at a position suitable for performing the actual measurement on the basis of results of the preliminary measurement; and

performing the actual measurement when it is determined that the measurement object is at a position suitable for performing the actual measurement on the basis of results of the preliminary measurement,

each of the above operations being carried out by an optical coherence tomography apparatus.

### Supplementary Note 6

The imaging method according to Supplementary note 5, wherein, in the preliminary measurement, the optical coherence tomography apparatus scans a range narrower than a range of scanning in the actual measurement.

### Supplementary Note 7

The imaging method according to Supplementary note 5 or 6, wherein in the preliminary measurement, the optical coherence tomography apparatus performs scanning at a higher speed than a speed of scanning in the actual measurement.

### Supplementary Note 8

The imaging method according to any one of Supplementary notes 5 to 7, wherein in the preliminary measurement, the optical coherence tomography apparatus performs scanning along a trajectory with a smaller change in speed than a change in speed of scanning in the actual measurement.

### Supplementary Note 9

A non-transitory computer readable medium storing an imaging program causing an optical coherence tomography apparatus to execute:

processing of performing preliminary measurement by emitting a light beam swept in a wavelength sweep range narrower than a wavelength sweep range in actual measurement;

processing of determining whether a measurement object is at a position suitable for performing the actual measurement on the basis of results of the preliminary measurement; and

processing of performing the actual measurement when it is determined that the measurement object is at a position suitable for performing the actual measurement on the basis of results of the preliminary measurement.

### Supplementary Note 10

The non-transitory computer readable medium storing the imaging program according to Supplementary note 9, the program causing the optical coherence tomography apparatus to execute, in the preliminary measurement, processing of scanning a range narrower than a range of scanning in the actual measurement.

### Supplementary Note 11

The non-transitory computer readable medium storing the imaging program according to Supplementary note 9 or 10, the program causing the optical coherence tomography apparatus to execute, in the preliminary measurement, processing of scanning at a higher speed than a speed of scanning in the actual measurement.

### Supplementary Note 12

The non-transitory computer readable medium storing the imaging program according to any one of Supplementary notes 9 to 11, the program causing the optical coherence tomography apparatus to execute, in the preliminary measurement, processing of scanning along a trajectory with a smaller change in speed than a change in speed of scanning in the actual measurement.

Although the present disclosure has been described above with reference to the example embodiments, the present disclosure is not limited by the above. Various modifications that can be understood by a person skilled in the art within the scope of the disclosure can be made to the configurations and details of the present disclosure.

### INDUSTRIAL APPLICABILITY

There are provided an optical coherence tomography apparatus 100, an imaging method, and a non-transitory computer readable medium storing an imaging program capable of detecting the position of a measurement object more promptly and performing measurement at appropriate timing.

### REFERENCE SIGNS LIST

### 100 OPTICAL COHERENCE TOMOGRAPHY APPARATUS

### 101 WAVELENGTH SWEEPING LASER LIGHT SOURCE

### 102 CIRCULATOR

### 103 BRANCHING AND MERGING DEVICE (BRANCHING UNIT, MERGING UNIT)

### 104 FIBER COLLIMATOR

### 105 IRRADIATION OPTICAL SYSTEM (IRRADIATION UNIT)

### 106 REFERENCE LIGHT MIRROR

### 107 BALANCED PHOTODETECTOR (MEASUREMENT UNIT)

### 108 OPTICAL SPECTRUM DATA GENERATION UNIT (MEASUREMENT UNIT)

### 109 CONTROL UNIT

### R1, R3 OBJECT LIGHT BEAM

### R2, R4 REFERENCE LIGHT BEAM

### R5, R6 INTERFERENCE LIGHT BEAM

### Z1, Z2 MEASUREMENT RANGE

### 200 MEASUREMENT OBJECT

What is claimed is:

1. An optical coherence tomography apparatus comprising:
a wavelength sweeping laser light source;
a branching unit configured to branch a light beam emitted from the wavelength sweeping laser light source into an object light beam and a reference light beam;
an irradiation unit configured to apply the object light beam output from the branching unit to different positions on a surface of a measurement object;
a merging unit configured to cause interference of the object light beam scattered from the measurement object with the reference light beam and generate a plurality of interference light beams;
a measurement unit configured to generate information about wavelength dependence of an intensity difference between the plurality of interference light beams output from the merging unit; and
a control unit configured to acquire structural data of the measurement object in a depth direction on the basis of the information about wavelength dependence of an intensity difference between the plurality of interference light beams generated by the measurement unit, control the irradiation unit to acquire a plurality of pieces of the structural data in the depth direction while moving a plurality of irradiation positions of the object light beam along a direction orthogonal to the depth direction of the measurement object, and connect the acquired plurality of pieces of the structural data in the depth direction, wherein
preliminary measurement is performed by causing the wavelength sweeping laser light source to emit a light beam swept in a wavelength sweep range narrower than a wavelength sweep range in actual measurement,
the control unit determines whether the measurement object is at a position suitable for performing the actual measurement on the basis of results of the preliminary measurement, and
the actual measurement is performed when the control unit determines that the measurement object is at a position suitable for performing the actual measurement on the basis of results of the preliminary measurement.

2. The optical coherence tomography apparatus according to claim 1, wherein in the preliminary measurement, the irradiation unit scans a range narrower than a range of scanning in the actual measurement.

3. The optical coherence tomography apparatus according to claim 1, wherein in the preliminary measurement, the irradiation unit performs scanning at a higher speed than a speed of scanning in the actual measurement.

4. The optical coherence tomography apparatus according to claim 1, wherein in the preliminary measurement, the irradiation unit performs scanning along a trajectory with a smaller change in speed than a change in speed of scanning in the actual measurement.

5. An imaging method comprising:
performing preliminary measurement by causing a wavelength sweeping laser light source to emit a light beam swept in a wavelength sweep range narrower than a wavelength sweep range in actual measurement;
determining whether a measurement object is at a position suitable for performing the actual measurement on the basis of results of the preliminary measurement; and
performing the actual measurement when it is determined that the measurement object is at a position suitable for performing the actual measurement on the basis of results of the preliminary measurement,
each of the above operations being carried out by an optical coherence tomography apparatus.

6. The imaging method according to claim 5, wherein, in the preliminary measurement, the optical coherence tomography apparatus scans a range narrower than a range of scanning in the actual measurement.

7. The imaging method according to claim 5, wherein in the preliminary measurement, the optical coherence tomography apparatus performs scanning at a higher speed than a speed of scanning in the actual measurement.

8. The imaging method according to claim 5, wherein in the preliminary measurement, the optical coherence tomography apparatus performs scanning along a trajectory with a smaller change in speed than a change in speed of scanning in the actual measurement.

9. A non-transitory computer readable medium storing an imaging program causing an optical coherence tomography apparatus to execute:
processing of performing preliminary measurement by emitting a light beam swept in a wavelength sweep range narrower than a wavelength sweep range in actual measurement;
processing of determining whether a measurement object is at a position suitable for performing the actual measurement on the basis of results of the preliminary measurement; and
processing of performing the actual measurement when it is determined that the measurement object is at a position suitable for performing the actual measurement on the basis of results of the preliminary measurement.

10. The non-transitory computer readable medium storing the imaging program according to claim 9, the program causing the optical coherence tomography apparatus to execute, in the preliminary measurement, processing of scanning a range narrower than a range of scanning in the actual measurement.

11. The non-transitory computer readable medium storing the imaging program according to claim 9, the program causing the optical coherence tomography apparatus to execute, in the preliminary measurement, processing of scanning at a higher speed than a speed of scanning in the actual measurement.

12. The non-transitory computer readable medium storing the imaging program according to claim 9, the program causing the optical coherence tomography apparatus to execute, in the preliminary measurement, processing of scanning along a trajectory with a smaller change in speed than a change in speed of scanning in the actual measurement.
